SEQUENCE LISTING (22 sequences)

SEQ ID NO: 1 - protein, 3 aa, Artificial Sequence
  Synthetic
YID

SEQ ID NO: 2 - protein, 3 aa, Artificial Sequence
  Synthetic
RWD

SEQ ID NO: 3 - protein, 3 aa, Artificial Sequence
  Synthetic
DVR

SEQ ID NO: 4 - protein, 7 aa, Artificial Sequence
  Synthetic
RESXNVA

SEQ ID NO: 5 - protein, 7 aa, Artificial Sequence
  Synthetic
ESXPRQA

SEQ ID NO: 6 - protein, 7 aa, Artificial Sequence
  Synthetic
VARENIA

SEQ ID NO: 7 - protein, 7 aa, Artificial Sequence
  Synthetic
RWEREDA

SEQ ID NO: 8 - protein, 6 aa, Artificial Sequence
  Synthetic
EWWETV

SEQ ID NO: 9 - protein, 7 aa, Artificial Sequence
  Synthetic
SVYQLDA

SEQ ID NO: 10 - protein, 7 aa, Artificial Sequence
  Synthetic
XHEFYGA

SEQ ID NO: 11 - protein, 7 aa, Artificial Sequence
  Synthetic
HEXXLVA

SEQ ID NO: 12 - protein, 7 aa, Artificial Sequence
  Synthetic
SSXKKDA

SEQ ID NO: 13 - protein, 7 aa, Artificial Sequence
  Synthetic
RXDKEAA

SEQ ID NO: 14 - protein, 7 aa, Artificial Sequence
  Synthetic
FQGTREA

SEQ ID NO: 15 - protein, 7 aa, Artificial Sequence
  Synthetic
TGTNRYA

SEQ ID NO: 16 - protein, 7 aa, Artificial Sequence
  Synthetic
KWATRYA

SEQ ID NO: 17 - protein, 7 aa, Artificial Sequence
  Synthetic
NSTKFDA

SEQ ID NO: 18 - protein, 7 aa, Artificial Sequence
  Synthetic
EHATYRA

SEQ ID NO: 19 - protein, 3 aa, Artificial Sequence
  Synthetic
FPK

SEQ ID NO: 20 - protein, 3 aa, Artificial Sequence
  Synthetic
HWK

SEQ ID NO: 21 - protein, 3 aa, Artificial Sequence
  Synthetic
WEE

SEQ ID NO: 22 - protein, 3 aa, Artificial Sequence
  Synthetic
LLR

(19) United States
(12) Patent Application Publication
(10) Pub. No.: US 2005/0032138 A1
(43) Pub. Date: Feb. 10, 2005

(54) Method for identifying ligands specific for structural isoforms of proteins

(76) Inventors: Julia T. Lathrop, Falls Church, VA (US); David J. Hammond, Laytonsville, MD (US); Larisa Cervenakova, Rockville, MD (US); Liliana Gheorghiu, Cambridge, MA (US); Oksana Yakovleva, Derwood, MD (US)

Correspondence Address: JOHN S. PRATT, ESQ;KILPATRICK STOCKTON, LLP, 1100 PEACHTREE STREET, ATLANTA, GA, 30309, US

(21) Appl. No.: 10/823,888

(22) Filed: Apr. 14, 2004

Related U.S. Application Data
(60) Provisional application No. 60/462,658, filed on Apr. 14, 2003.

(51) Int. Cl. G01N 33/53; G01N 33/537; G01N 33/543

(52) U.S. Cl. 435/7.92

(57) ABSTRACT

A method of identifying a ligand specific for a structural isoform of a protein by the binding of the structural isoform to a ligand on a support, a direct positional transfer of the structural isoform and a control isoform between two or more different solid or semi-solid supports and detection of at least one of the isoforms on each support, thus allowing for subtractive identification techniques to be used to identify the subset of ligands, or a ligand, specific for the structural isoform.

### CROSS-REFERENCE TO RELATED APPLICATIONS

The present application claims the benefit of U.S. Provisional Patent Application No. 60/462,658 filed Apr. 14, 2003, incorporated herein by reference in its entirety.

### FIELD OF THE INVENTION

The invention relates generally to methods for identifying ligands having binding specificity for a protein isoform.

### BACKGROUND

The assembly and misassembly of normally soluble proteins into conformationally altered, insoluble aggregates is thought to be a causative process in a variety of diseases. Examples of some insoluble proteins and their associated diseases include, but are not limited to, the β-peptide in Alzheimer's disease and cerebral amyloid angiopathy; α-synnuclein deposits in the Lewy bodies of Parkinson's disease; tau in neurofibrillary tangles in frontal temporal dementia and Pick's disease; superoxide dismutase in amyotrophic lateral sclerosis; and huntingtin in Huntington's disease. Abnormal self-assembly of human transthyretin into amyloid fibrils causes two forms of human disease, namely senile systemic amyloidosis and familial amyloid polyneuropathy. A conformational change in prion protein structure appears to be involved in the neurodegenerative process of transmissible spongiform encephalopathies (TSEs) such as Creutzfeldt-Jakob disease.

Often, these highly insoluble proteins form aggregates composed of fibrils with a characteristic β-pleated sheet conformation. In the central nervous system (CNS), amyloid can be present in cerebral and meningeal blood vessels (cerebrovascular deposits) and in brain parenchyma (plaques). A precise mechanism by which neuritic plaques are formed and the relationship of aggregate formation to the disease-associated neurodegenerative processes are largely unknown.

Native or cellular prion protein, “PrPc”, is widely distributed throughout the Mammalia and has a particularly well-conserved amino acid sequence and protein structure. Infectious prions are thought to be composed of a modified form of the normal cellular (PrPc) prion protein and have been referred to as “PrPsc” (indicating the scrapie form of the protein); as “PrPcjd” (indicating the CJD form of the protein); and as “PrPres” (indicating the proteinase K (PK)-resistant form of the protein). Prions have some properties in common with other infectious pathogens, but do not appear to contain a nucleic acid. Instead, it has been proposed that a post-translational conformational change is involved in the conversion of non-infectious PrPc into infectious PrPsc, during which α-helices are transformed into β-sheets. PrPc contains three α-helices and has little β-sheet structure; in contrast, PrPsc is rich in β-sheet. The conversion of PrPc to PrPsc is believed to lead to the development of transmissible spongiform encephalopathies (TSEs) during which PrPsc accumulates in the central nervous system and is accompanied by neuropathologic changes and neurological dysfunction. An infectious form of the prion protein is considered necessary and possibly sufficient for the transmission and pathogenesis of these transmissible neurodegenerative diseases of animals and humans. (See Prusiner 1991 Science 252: 1515-1522).

Examples of TSE diseases affecting animals include, but are not limited to, scrapie in sheep, bovine spongiform encephalopathy (BSE) or “mad cow disease” in cattle, transmissible mink encephalopathy (TME), and chronic wasting disease (CWD) in deer and elk. The spectrum of human TSE diseases includes, but is not limited to, kuru, Creutzfeldt-Jakob disease (CJD), Gerstmann-Straussler-Scheinker (GSS) disease and fatal familial insomnia. Recently, evidence has developed that BSE is transmissible to a wide range of other mammals including humans. The human form of this disease is referred to as variant CJD (vCJD).

Methodologies that can readily separate or that can distinguish between two or more different conformational forms of a protein, such as PrPc and PrPsc, are needed to understand the process of conversion and to find structures that specifically interact with the disease-associated forms. Current methodologies for separating or distinguishing between isoforms include differential mobility in polyacrylamide gels in the presence of a chaotrope, such as urea, particularly, transverse urea gradient (TUG) gels; differential sensitivity to protease treatment, such as PK treatment, and the detection of the PK-resistant digest product of PrPsc referred to as PrPres; differential precipitation by Na-phosphotungstate; differential temperature stability; relative solubility in non-ionic detergents; and the ability for fibrillar structures to bind certain chemicals, such as Congo red and isoflavin S. There remains a need to identify high affinity ligands or reagents that are specific for the conformationally altered protein, especially forms associated with disease. Such ligands or reagents are useful for a variety of uses, including, but not limited to, developing possible diagnostic kits; separation and purification of the different forms of protein; removal of infectious forms of the disease from therapeutic agents, biological products, vaccines and foodstuffs, and for therapy.

### SUMMARY

Methods for the identification of ligands that are specific for a structural isoform of a protein, also referred to as a target structural isoform, are provided herein. These ligands are used, for example, to separate, concentrate, or differentiate between structural isoforms of proteins and other targets in a sample, solution or a complex mixture. In a preferred embodiment, the protein is a prion protein and the structural isoform is an infectious prion isoform.

In accordance with an embodiment of the method according to certain aspects of the present invention, one or more immobilized ligands are contacted with a sample containing a target protein isoform under conditions sufficient, or allowing, to cause formation of a ligand-isoform complex. The ligand or the ligand-isoform complex is immobilized on or in a first support.

In another embodiment, prior to immobilization on the first support, a library of test ligands is immobilized on a solid phase, such as but not limited to, polymeric beads, resulting in a plurality of beads bearing different ligands, with multiple copies of a single, unique test ligand present on the surface of the bead. These beads are subsequently immobilized on or in a first support. In this manner, the ligand is indirectly immobilized on the first support.

Alternatively, the test ligands are immobilized by direct coupling to the first support, such as a membrane or a gel. For example, a ligand library is immobilized on a first support, such as a two-dimensional array, where each species of test ligand is placed at a unique position within the array. A protein isoform is thereby captured at a unique position in the array based on its interaction with a specific test ligand.

Ligand-isoform complexes are detected following immobilization of the complexes on the first support. Detection is associated directly with a ligand-isoform complex, such as an on-bead detection, or indirectly, such as a capture of a chemiluminescent signal on an x-ray film.

The isoforms are then transferred to a second support and immobilized thereupon such that they are present in positions that correspond to the positions of immobilization on the first support. Preferably, the isoforms are separated from the ligands and then immobilized on the second support, leaving the test ligands bound to the first support. The isoforms immobilized on the second support are then detected. In a preferred embodiment, both a target isoform and a control isoform, differing from the target isoform in the folding pattern or other secondary or tertiary structure, are immobilized on the second support, and the target isoform is modified prior to the second detection event. The target isoform may be modified by any means known to those of skill in art, but is preferably modified by denaturation or enzymatic cleavage to form a different isoform of the same protein as the target isoform. In one embodiment, both the modified target isoform and the control isoform are detected on the second support using a detection marker.

The detection patterns on the first and second support are then aligned and compared. First, a determination is made of the location of the target isoform on the second support. In one embodiment, the location is indicated by the presence of a detection signal on the second support and the absence of a corresponding detection signal on the first support or visa versa. Thus, the first detection identifies either a subset of isoforms or all of the isoforms and the second detection identifies the subset of isoforms not detected in the first step and vice versa. The term “subset” as used herein in reference to protein isoforms denotes a group of isoforms of a protein. The subset comprises from zero to all of protein isoforms. By aligning the first and second supports and analyzing, or comparing, the detection results, the ligands to which the various subsets of isoforms were initially bound may be detected, identified and isolated. That is, once the unique position of the protein is identified on the second support, its former position on the first support (where it was captured by the ligand) can be determined, leading to the identification and isolation of the ligand responsible for its original capture.

The method described herein offers a number of advantages over currently available methods for identifying ligands for the separation of protein structural isoforms. First, a protein and its cognate ligand may be identified after their dissociation. Both the protein and its ligand are identified without the necessity for prior modification of either, such as, but not limited to, by labeling with fluorescent molecules, radioactive amino acids or molecules, biotinylation, and antibody derivatization. Thus, when detection follows transfer, interaction is completely avoided between the components of the detection system and the ligand, supports, or other elements of the system. Second, because the detection methods may be separated in time and space, they do not interfere with each other, and can be designed to detect different populations of the isoforms. Third, methods that require denaturation or inactivation may be employed in the same procedure as methods that maintain biological activity, and the ability of the proteins to identify the ligand to which they were originally bound can also be maintained. Finally, ligands that differentiate between multiple forms of the protein can be identified and used to separate, purify, concentrate, or diagnose, or any combination thereof, the presence of different structural forms, or isoforms, of the protein. None of these advantages are realized by comparable technologies presently available.

### BRIEF DESCRIPTION OF THE DRAWINGS

FIG. 1 is a schematic showing transfer of the target and control isomers from beads embedded in an agarose gel (first support) to a membrane (second support).

FIG. 2 is a schematic showing a screening method for PrPsc specific ligands, wherein non-denatured PrPc isomers are detected on a first support, denatured PrPsc and PrPc isoforms are detected on a second support, and wherein the second support detection results are compared with the first support agarose gel containing fast-red stained beads.

FIG. 3 is a flow chart schematically representing a method of identifying PrPsc ligands according to certain preferred embodiment of the present invention.

### DETAILED DESCRIPTION

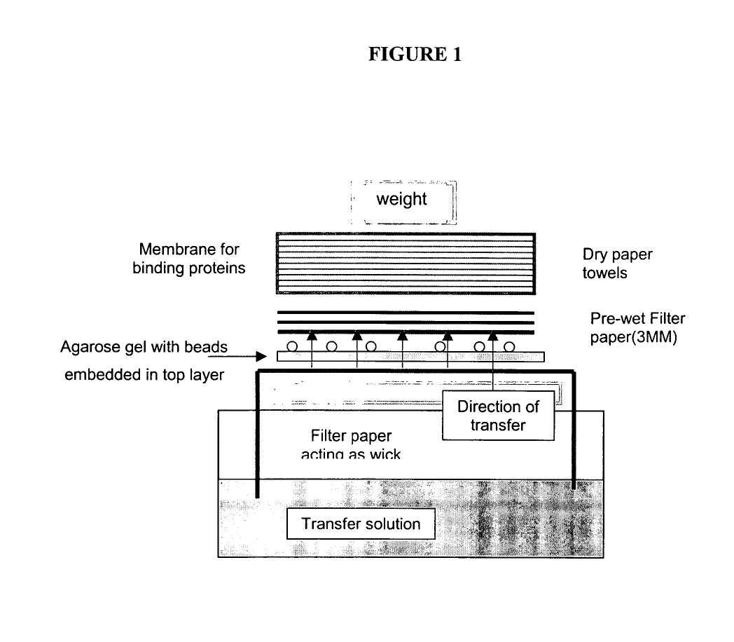

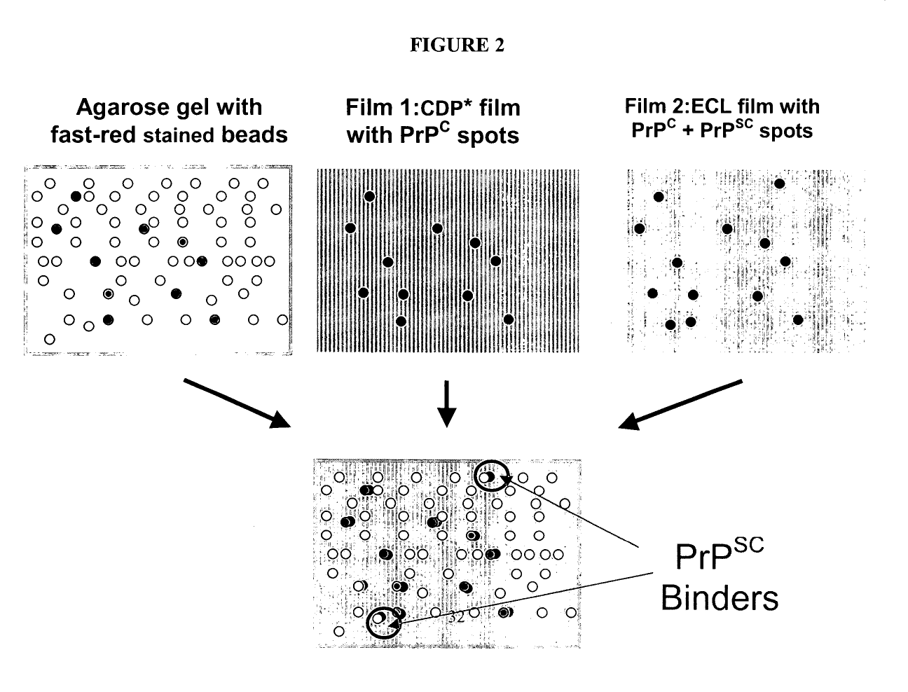

Methods for identifying ligands specific for, or having binding specificity for, a structural isoform of a protein are described herein. The methods generally include binding a target structural isoform to a test ligand to form an isoform-ligand complex, wherein the ligand or complex is immobilizedon a first support, detecting bound isoform on the first support, transferring the isoform to a second support by direct positional transfer, and detecting the isoform on the second support, thus allowing for subtractive identification techniques to be used to identify ligands specific for the target structural isoform. FIGS. 1 and 2 show representative non-limiting examples of methods for transferring the isoforms between the one or more supports and the subtractive identification techniques.

The ligand or complex is immobilized on the first support in a variety of ways known to those skilled in the art. For example, the ligand is immobilized in or on the first support directly or indirectly. The term “on”, when referring to the attachment of a ligand to a support, includes attachment of the ligand to the exterior or a surface of the support as well as embedding the ligand within the support. In one embodiment, the first support is a solid or semi-solid substance, such as a gel, that hardens or solidifies upon polymerization. In this embodiment, the first support contains a solid phase dispersed therein to which the ligand is attached. For example, in a preferred embodiment, the solid phase is a particle, such as a polymeric bead, which is coated with bound ligand. In this way, a higher concentration of ligand can be maintained in a particular location on the support. Alternatively, the ligand is attached directly to the support, such as in a one or two-dimensional array or matrix, by coupling means known to those skilled in the art.

The ligand is contacted with a sample containing the target isoform of interest, thereby creating an isoform-ligand complex, either before or after the ligand is immobilized on the first support. Optionally, a control isoform is also immobilized on the first support. As used herein, the term “control isoform” refers to a protein having the same amino acid sequence as the target isoform, but differs in its folding pattern or other secondary or tertiary structure. The target isoform, the control isoform, or both the target isoform and the control isoform may be detected on the first support.

Subsequently, the target isoform and, optionally, the control isoform, are transferred to a second support, such as, but not limited to, a membrane, to achieve direct positional transfer of the isoforms from the first support to the second support. Either the target isoform, the control isoform, or both are detected on the second support to allow for alignment of the first support and the second support and determination of the location of the ligand that bound to the target isoform on the first support using subtractive identification techniques. In a preferred embodiment, the target isoform is modified before the second detection step.

The target and control structural isoform proteins described herein include isoforms of any protein having more than one structural isoform including, but not limited to, a prion protein isoform; a β-peptide isoform as involved in Alzheimer's disease and cerebral amyloid angiopathy; an α-synnuclein isoform; a tau protein isoform as involved in neurofibrillary tangles in frontal temporal dementia and Pick's disease; a superoxide dismutase isoform; a huntingtin isoform; and a human transthyretin isoform protein. In one embodiment, the structural isoform protein is an infectious or disease-causing isoform. In another embodiment the structural isoform protein is a prion protein such as, but not limited to, PrPc, PrPsc or PrPres.

Definitions

The terms “a,” “an” and “the” as used herein are defined to mean “one or more” and include the plural unless the context is inappropriate.

The terms “protein”, “peptide” “polypeptide” and “oligopeptide” are used interchangeably and are defined herein as a chain of amino acids in which carbons are linked through peptide bonds formed by a condensation reaction between the carboxyl group of one amino acid and the amino group of another amino acid.

The term “PrPc” refers to a native prion protein molecule, which is naturally and widely expressed within the body of the Mammalia. Its structure is highly conserved and is not associated with a disease state.

The term “PrPsc” refers to a conformationally altered form of the PrPc molecule that is believed by those skilled in the art to be infectious and is associated with diseases such as, but not limited to, TSE/prion diseases, including vCJD, CJD, kuru, fatal insomnia, GSS, scrapie, BSE, CWD, and other TSEs of captive and experimental animals. PrPsc has the same amino acid sequence as normal, cellular PrPc, but has converted some of the α-helix to β-pleated sheet and is associated with a disease state. Accordingly, the term “PrPsc” encompasses the forms of the prion protein referred to as the “PrPtse” and “PrPcjd” forms.

The term “PrPres” refers to proteinase resistant derivatives of the PrPsc protein of 27-30 kDa that remain following partial digestion of PrPsc with PK.

The term “PrPr” refers to a prion protein expressed by recombinant technology.

The term “PrP” refers to a prion protein in general.

The term “specific for” or “having binding specificity for”, which can be used interchangeably with the term “cognate”, when referring to a ligand, means a ligand that binds to a target protein with sufficient affinity and avidity to result in the production of a ligand-target protein complex.

The term “structural isoforms” refers to forms of proteins that differ only in their folding pattern or other secondary or tertiary structure, but have the same primary amino acid sequence.

The term “3F4” refers to the monoclonal antibody specific to native forms of PrPc, but not native PrPsc or PrPres. The antibody has specificity for denatured forms of hamster and human PrPc, PrPsc and PrPres.

Ligands

The term “ligand” refers to a molecule to which a protein binds, including, but not limited to, a small molecule, a peptide, a protein, a polysaccharide or a nucleic acid. Preferred test ligands are peptides, particularly peptides of 1 to about 15 amino acid residues. Peptide ligands can be produced by techniques that are used to make a combinatorial library such as “split, couple, recombine” methods as well as by other approaches described in the literature. See, for example, Furka et al, Int. J. Peptide Protein Res., 37, 487-493 (1991); K. S. Lam et al., Nature, 354, 82-84 (1991); PCT Publication WO 92/00091; U.S. Pat. No. 5,133,866; U.S. Pat. No. 5,010,175; U.S. Pat. No. 5,498,538. Expression of peptide libraries is described by Devlin et al., Science, 249, 404-406 (1990). Using methods known to one of skill in the art, vast libraries of ligands can be synthesized by a series of coupling reactions directly onto a bead that may be later immobilized on a first solid support, or synthesized on a bead, cleaved and then attached to the first solid support, or synthesized directly onto the first solid support. Typically, the ligands are synthesized on beads such that multiple copies of a single ligand are synthesized on each bead. One of skill in the art will also appreciate that the ligands may be attached to the bead or first solid support by covalent attachment, directly or through a linker molecule.

Supports

As stated above, the methods described herein include a direct positional transfer of a target isoform between two or more supports to allow for differential detection of the isoform on each support and subsequent identification of a ligand having binding specificity for an isoform using subtractive identification techniques. In one embodiment, a first support and a second support are employed. The term “support” refers to any material in or on which the ligand or isoform is immobilized. The isoform may be attached to a ligand immobilized on the support, or the isoform itself may be immobilized on the support. For example, it is preferable that the ligand is immobilized on the first support, and the isoform is bound to the immobilized ligand, but that only the isoform (not the ligand) is transferred to and immobilized on the second support. The term “immobilized” refers to both a temporary and a semi-permanent retainment of a molecule in a particular position on a support. The isoforms are temporarily immobilized on a support until their transfer to another support, and the ligands are preferably semi-permanently immobilized on a support so that transfer of the isoform does not also affect transfer of the ligand.

Although a preferred first support is a gel, such as an agarose gel, containing a solid phase substance, such as polymeric beads, the first support may also include any material onto which the ligands are directly coupled to form an array. The term “array” is used herein to denote a spatial arrangement, such as an arrangement of molecules on a solid support, and includes a one dimensional arrangement, a two dimensional arrangement, a three dimensional arrangement, a circular arrangement or any modification or variation thereof. A variety of porous matrices are useful as first support materials including, but not limited to, synthetic polymers, such as polyacrylamides, gelatins, lipopolysaccharides, and silicates. The first support may also be composed of glass, nitrocellulose, silicon, or polyvinyldifluoride nylon.

When a bead, or particle, is used as a component of the first support, the ligand is attached to the bead in any manner provided above. The bead may be of any material capable of forming a particle including, but not limited to, acrylic, polyacrylamide, polymethacrylate, polystyrene, dextran, agarose, celluloses, polysaccharides, hydrophilic vinyl polymers, celite, sepharose, polymerized derivatives thereof, and combinations thereof. A particularly preferred bead material is a polyhydroxylated methacrylate polymer, and more preferably a Toyopearl™ 650-M amino resin (Tosoh BioScience, Montgomeryville, Pa.). Various other methacrylate polymer resins are commercially available and commonly employed in a bead form. It is to be understood that the ligand-bearing beads may be immobilized on or in the first support before, during, or after contact with the isoform protein-containing sample. It is to be further understood that the ligands may be directly attached to the support, directly synthesized on the support, and/or directly embedded within the support instead of first being attached to a bead.

In accordance with the methods described herein, the isoforms are transferred from the first support to a second support. The target isoform, and optionally the control isoform, are transferred between supports using any methods known to those of skill in the art, including, but not limited to, capillary action. Representative reagents for transferring the isoform to the second support include, but are not limited to, water, salt solutions, solutions containing denaturing agents such as guanidinium hydrochloride, organic solvents, compounds that specifically compete with the binding of at least one isoforms to the ligand, and other standard reagents for removing proteins from affinity ligands under conditions sufficient to remove at least one isoform from the ligand. A non-limiting example of the transfer is schematically depicted in FIG. 1. The term “second support” refers to any material capable of immobilizing the isoform protein following removal or elution from the first support. Second support materials include, for example, nitrocellulose, polyvinyl difluoride, nylon and cellulose membranes, glass and silicon. One or both of the target and control isoforms are detected following immobilization on the second support.

A non-limiting example of such transfer is schematically depicted in FIG. 1. The term “second support” refers to any material capable of immobilizing the isoform protein following removal or elution from the first support. Second support materials include, for example, nitrocellulose, polyvinyl difluoride, nylon and cellulose membranes, glass and silicon. One or both of the target and control isoforms are detected following immobilization on the second support.

Modification

In a preferred embodiment, the isoform is modified between the first detection step and the second detection step in order to change a detection characteristic. Such modification may occur before, during, or after transfer of the isoform from one support to another. Preferably, the modification of an isoform allows for the use of a single detection agent during both the first and second detection steps while still producing different first and second detection sets amenable to subtractive identification techniques.

Detection characteristics may be modified by denaturing or cleaving the isoform, by derivatizing the isoform with a label or a linker, by modifying or inactivating the enzymatic activity of the isoform, or by any other means known to those of skill in the art. In a preferred embodiment, the target isoform is a PrPsc that is denatured using a denaturing agent. Representative denaturing agents include guanidinium hydrochloride; urea; beta-mercaptoethanol; detergents; thiol reagents including sodium thiosulfate and dithiothreitol (DTT); sodium dodecyl sulfate (SDS), Tween, and Sarkosyl. Denaturation of the PrPsc allows for detection of the isoform on the second support by a detection marker such as the commercially available 3F4 monoclonal antibody. This particular antibody binds with specificity to both native and denatured forms of PrPc and to denatured forms of PrPsc, but does not bind to native, non-denatured PrPsc. An isoform-ligand complex immobilized on the first support would not be detected by the 3F4 monoclonal antibody, however, the modified isoform would be detected by the antibody when immobilized on the second support. A detection signal observed on the second support, but absent on the first support, would indicate the presence of the PrPsc isoform at the corresponding location on the first support. One could conclude that the ligand immobilized at that corresponding location on the first support binds with specificity to the PrPsc isoform.

Accordingly, in a preferred embodiment, identification of a ligand specific for a structural isoform of a protein is achieved by practicing the following: contacting a sample containing a target isoform with a test ligand under conditions sufficient to cause formation of a ligand-isoform complex; immobilizing the ligand-isoform complex and, optionally, a control isoform on a first support; detecting the isoform on the first support; transferring the isoform to a second support and immobilizing the isoform thereupon; detecting the isoform on the second support, wherein the detectability of the isoform is modified prior to detection; aligning the first and second supports and determining a location of the target isoform on the second support, wherein the location is indicated by the presence of a detection signal on the second support and the absence of a corresponding detection signal on the first support; determining a location of the target isoform on the first support; and identifying the ligand at that location.

In an alternative embodiment, differential detection between the first and the second supports is achieved by using different detection methods for the various isoforms present on each support. In the case of serpins such as alpha-1 protease inhibitor (API), both active and latent isoforms exist. API loses its activity when it “flips” its structure. Ligands that are specific for one of the isoforms may be identified by incubating them with the starting materials containing API. The ligands are then immobilized in a gel and incubated with an enzyme against which API has activity, such as porcine elastase. Ligands complexed with active API isoforms are identified via a colorimetric assay. The protein isoforms are subsequently transferred from the ligands under non-denaturing conditions to a second solid support, such as a membrane. The membrane is then incubated with a detection marker such as an antibody that detects all forms of API. It is possible that some ligands may bind both active and latent forms of API; however, with this method, ligands that bind only the active form of the protein are identified. Other embodiments, including identification of ligands that bind only certain forms of amyloid proteins may also be contemplated within the scope of this method.

In still other embodiments, differential detection between the first and second supports is achieved when only one of the target or control isoforms is transferred to and detected on the second support following detection of both the control and target isoforms on the first support. For example, PK can be used to digest control prion isoform (PrPc) and cleave PrPsc target isoform into PK-resistant fragments, known as PrPres, on the first support. Such treatment results in the transfer of only PrPres to the second support. A detection marker, such as a commercially available 3F4 antibody (available from Signet Laboratories, Inc., Dedham, Mass.), can then be used to detect PrPres on the second support. Alignment of the first and second support indicates the location of one or more test ligands specific for the PrPsc isoform.

Detection

In several of the detection methods described above, a detectable ligand, or marker, is used to determine the presence of a protein isoform. The terms “detectable marker” or “detection method” refer to entities or methods with which the presence of a protein can be determined. When employing a detectable marker, the particular label or detectable group used to detect the isoform is not critical as long as it is compatible with the requirements of the assay. The detectable label can be any material having a detectable physical or chemical property. Such detectable labels have been well-developed and, in general, any label useful in such methods can be applied to the present method. Thus, a label is any composition detectable by spectroscopic, photochemical, biochemical, immunochemical, electrical, optical or chemical means. Useful labels include fluorescent dyes (such as fluorescein isothiocyanate, Texas red, rhodamine, and the like), radiolabels (such as ³H, ¹²⁵I, ³⁵S, ¹⁴C, or ³²P), enzymes (such as LacZ, CAT, horseradish peroxidase, alkaline phosphatase and others, commonly used as detectable enzymes, either in an enzyme immunoassay (EIA) or in an enzyme-linked immunosorbent assay (ELISA), and colorimetric labels such as colloidal gold or colored glass or plastic (such as. polystyrene, polypropylene, latex, etc.) beads. The label may be coupled directly or indirectly to the desired component of the assay according to methods well known in the art. As indicated above, a wide variety of labels may be used, with the choice of label depending on the sensitivity required, ease of conjugation of the compound, stability requirements, available instrumentation, appropriateness to the assay, and disposal provisions.

Non-radioactive labels are often attached by indirect means. Generally, a secondary ligand molecule (such as biotin) is covalently bound to the first ligand. The secondary ligand then binds to a tertiary ligand (such as streptavidin) molecule which is either inherently detectable or covalently bound to a signal system, such as a detectable enzyme, a fluorescent compound, or a chemiluminescent compound. A number of secondary and tertiary ligands can be used. Where a secondary ligand has a natural tertiary ligand, for example, biotin, thyroxine, or cortisol, it can be used in conjunction with the labeled, naturally occurring tertiary ligands. Alternatively, any haptenic or antigenic compound can be used in combination with an antibody.

The secondary ligands can also be conjugated directly to signal generating compounds, such as by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, such as luminol.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, such as by microscopy, visual inspection, via photographic film, by the use of electronic detectors such as charge coupled devices (CCDs) or photomultipliers and the like. Similarly, enzymatic labels are detected by providing appropriate substrates for the enzyme and detecting the resulting reaction product. Finally, simple colorimetric labels may be detected simply by observing the color associated with the label.

It is to be understood that a combination of different detectable markers may be employed in this method to accomplish the differentiation of the isoforms. The detectable markers of the present invention can be any molecular or biological entity that interacts with various isoforms in different ways. For example, the marker may be an enzyme or antibody that specifically interacts with one or several isoforms, a nucleic acid sequence which binds to one or several isoforms through hybridization, or a molecular entity that undergoes a detectable chemical reaction in the presence of one or several isoforms. Similarly, the marker can be specific for a protein that is complexed with other biological entities such as co-factors or enzymes. Alternatively, the protein itself may be detected directly by a spectral signal, including fluorescence, or by a molecular weight or protein sequence, through mass-spectrometry, or other means.

Detection of an isoform may also be achieved by detection of a biological, biochemical, or chemical activity of the isoform itself. It is an advantage of the present invention that the protein can be transferred to another support using conditions under which it retains its biological activity. For example, one isoform may retain or acquire an activity not present in a different isoform, and this activity used to differentiate between ligands that discriminate between isoforms.

Samples

The protein isoforms for use in the method described herein may be contained within numerous different types of samples including environmental and biological samples. The isoforms may co-exist in a purified, semi-pure, or in a complex environment within the sample. Environmental samples include, but are not limited to, water from a source such as a lake, ocean, stream, river, aquifer, well, water treatment facility or recreational water. In some embodiments of the invention, the sample contains synthetic target isoforms, including synthetic isoform peptides, recombinant isoform proteins, synthetic nucleic acid isoform species, combinatorial isoform libraries, organic solvents, extracts from soils, food, air and water supplies, swabs of environmental surfaces, and the like.

Examples of biological samples that may contain the protein isoforms include, but are not limited to, whole blood, blood-derived compositions or components, sera, cerebrospinal fluid, urine, saliva, milk, ductal fluids, tears, semen, or may be organ-derived, including brain or spleen, compositions from humans or animals, tissue homogenates, cell homogenates, conditioned media, fermentation broths, antibody preparations, plant homogenates and extracts, and food, including nutritional supplements. Other biological samples include those that contain collagen or gland extracts. As used herein, the terms “blood-derived compositions” and “blood compositions” are used interchangeably and are meant to include whole blood, red blood cell concentrates, plasma, platelet rich and platelet poor fractions, plasma precipitates, plasma supernatants, intravenous immunoglobulin preparations including IgA, IgE, IgG and IgM; purified coagulation factor concentrates; serpins, including α-1 protease inhibitor, anti-thrombin III, α₂ antiplasmin; fibrinogen concentrate, and albumin; or other various other compositions which are derived from human or animal. The term also includes purified blood derived proteins prepared by any of various methods common in the art including ion exchange, affinity, gel permeation, and/or hydrophobic chromatography or by differential precipitation.

Biological samples containing the isoforms of the present invention further include food products or nutritional supplements for either animal or human consumption. For example, the biological sample may contain material derived from any animal, including, but not limited to, a bovine; ovine; porcine; equine; murine, such as a mouse and a hamster; and a Cervidae, such as deer and elk, animal. The term “animal-derived materials” refers to the materials described above as well as materials containing animal parts such as muscle, connective tissue and/or organ tissue. Animal-derived materials further include, but are not limited to, bone meal, beef, beef by-products, sheep, sheep by-products, elk, elk by-products, pork, pork-by products, sausage, hamburger, baby food, gelatin, jelly, milk, and infant formula.

Identification of a PRPSC Specific Ligand

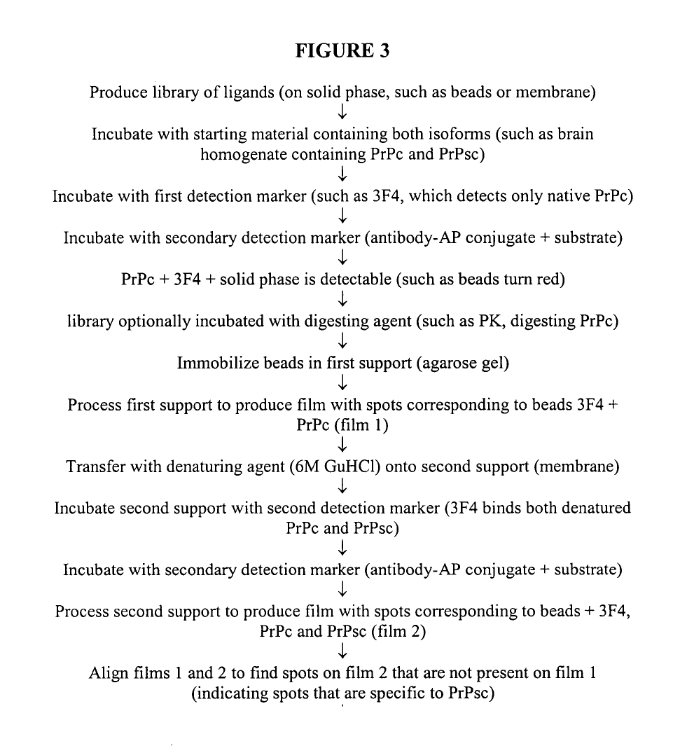

A preferred method for the identification of a ligand specific for the prion isoform PrPsc, and not PrPc, is described herein with reference to FIG. 3. In one embodiment, a complex sample containing both PrPc and PrPsc is incubated with a library of combinatorially-generated ligands that have been synthesized on chromatography resin beads such that each bead contains millions of copies of a single, unique ligand, and each bead bears a different ligand. Preferably, the sample is a brain homogenate from hamsters that have been infected with the scrapie. This brain homogenate contains both the normal cellular form of the prion protein, PrPc, and the infectious form, PrPsc. Alternatively, the sample is a brain homogenate from a human infected with sporadic Creutzfeldt-Jakob disease (CJD) or a brain homogenate from a patient infected with variant CJD (vCJD).

The sample is incubated with the library on beads for a period of time sufficient for the protein isoforms to bind to the various ligands via highly specific affinity interactions. Non-bound and weakly bound proteins are removed by washing. The bound proteins are detected by a first detection method, using a detection marker specific for PrPc. A preferred detection marker is the monoclonal antibody designated 3F4 (Signet Laboratories, Inc., Dedham, Mass.). This antibody can detect PrPc in its native and denatured forms; however, it can only detect PrPsc when it is denatured. The beads bearing ligands, on which the proteins are fractionated, are incubated with the detection marker. Bound detection marker is detected using a secondary detection marker such as a detectable antibody that binds to the first detection marker. Preferably, the secondary detection marker is an antibody conjugated to alkaline phosphatase (AP), which forms an insoluble, colored precipitate that stains those beads bearing the secondary antibody red upon reaction with an AP substrate. Thus, red beads indicate the presence of ligands to which PrPc or the detection marker or the secondary antibody has bound.

In one embodiment, the entire library that has been incubated with the starting material is then incubated with PK, which preferentially digests PrPc. This removes PrPc from the beads, leaving only PrPres for transfer and detection. In this and other embodiments, the treated library may then be immobilized on a first support such as a gel, preferably as an agarose gel. This first support immobilizes the beads in a thin monolayer. In one embodiment, the first support and beads are incubated with a chemiluminescent substance such as chemiluminescent alkaline phosphatase substrate and subsequently exposed to radiographic film to produce a film with spots in the location of beads that bound PrPc-detection marker-secondary marker (film 1). In this and other embodiments the proteins bound to the beads are then transferred off the beads, such as in a capillary manner by diffusion of a transfer buffer in one direction through the gel, through the beads, and through a second support, such as a protein-binding membrane, on which the proteins that have been stripped off the beads are captured. They are captured in the same relative position on the second support that they were immobilized in the first support. In one embodiment, the transfer buffer is a modifying agent, preferably denaturing, such as 6M Guanidine HCl (GuHCl), which removes and denatures proteins, dissociating them from the beads and maintaining them in a denatured state during the transfer.

The second support, to which the proteins are bound, is removed from the first support and processed. In one embodiment, bound, denatured PrPc and PrPsc (PrPres if the library has been PK treated) on a membrane are detected using a detection marker such as 3F4 antibody. Under the aforementioned conditions, 3F4 antibody allows for detection of both PrPc and PrPsc on the membrane, as they both are denatured. The bound 3F4 antibody is detected via a secondary antibody, such as an antibody conjugated to horseradish peroxidase (HRP). This enzyme is then detected via a chemiluminescent HRP substrate, and exposed to radiographic film. This incubation results in a film with spots indicating the presence of PrPc, PrPsc/PrPres and 3F4 antibody (film 2). Superimposition of films 1 and 2 indicates beads that have bound only 3F4 antibody (antibody binders), PrPc or 3F4 antibody, PrPc and PrPsc/PrPres (superimposable spots present on film 1 and 2), and that have bound only PrPsc/PrPres (spots present only on film 2). Alignment of film with the first support containing the beads enables the recovery of a specific bead with the desired characteristics.

Use of Ligands to Detect and Remove Structural Isoforms

The ligands identified using the methods described herein are antibody preparations, proteins, peptides, amino acids, nucleic acids, carbohydrates, sugars, lipids, organic molecules, polymers, and/or putative therapeutic agents, and the like. In a preferred embodiment, the ligands are peptide ligands. Ligands that are specific for structural isoforms or fragments of structural isoforms identified using the methods described above are useful for a variety of analytical, preparative, and diagnostic applications.

In one embodiment, the ligands identified using the methods provided herein are used for detecting the presence of structural isoforms in a biological fluid. The biological fluid, such as a test sample, is contacted with one or more ligands in accordance with the methods described herein under conditions sufficient to cause formation of a complex between the structural isoform and one or more of the ligands. The complex is then detected, thereby identifying the presence of the structural isoform in the biological fluid. The ligands identified by the methods described herein can also be used to detect isoform targets extracted into solution from a solid material. For example, a solid sample can be extracted with an aqueous or an organic solvent or a critical fluid, and the resultant supernatant can be contacted with the ligand. Examples of solid samples include plant products, particularly those that have been exposed to agents that transmit prions, such as bone meal derived from bovine sources; animal-derived products, particularly those that have been exposed to agents that transmit prions, such as bone meal derived from bovine sources. Other solid samples include brain tissue, corneal tissue, fecal matter, bone meal, beef by-products, sheep, sheep by-products, deer and elk, deer and elk by-products, and other animals and animal-derived products. Ligands in some embodiments can be used to detect the presence of structural isoforms in soil.

In another embodiment, ligands that bind structural isoforms are immobilized on a support, such as a bead or membrane, and used to bind and remove structural isoforms from a sample. Beads and membranes for removal of contaminants are well known in the art and described, for example, in Baumbach and Hammond (1992), Buettner (U.S. Pat. No. 5,834,318). In this embodiment, a biological sample is contacted with a structural isoform-binding ligand according to the invention under conditions sufficient to cause formation of a structural isoform-ligand composite or complex. The complex may than be removed from the biological sample, thereby removing the structural isoform from the biological sample. As indicated above, examples of biological samples include, such as blood, blood-derived compositions, plasma or serum. Additional biological fluids include cerebrospinal fluid, urine, saliva, milk, ductal fluid, tears or semen. Other biological fluids may contain collagen, brain and gland extracts.

Since the ligands identified using the methods described herein are specific for a particular isoform, ligands may be used for the selective concentration or removal of one of the isoforms over another. In some embodiments, the ligands distinguish between infectious and non-infectious isoforms, and these ligands may be used for the diagnosis and prognosis of diseases in a human or animal involving infectious or disease causing isoforms. Examples of diseases believed to be caused by a single isoform of a protein are prion-related diseases that include, but are not limited to, TSEs such as scrapie, which affects sheep and goats; BSE, which affects cattle; transmissible mink encephalopathy, feline spongiform encephalopathy and CWD of mule deer, white-tailed deer, black-tailed deer and elk; kuru, CJD, GSS, fatal insomnia and (vCJD), which affect humans.

The invention will be described in greater detail by way of specific examples. The following examples are offered for illustrative purposes, and are intended neither to limit nor define the invention in any manner.

### EXAMPLE 1

### Identification of Peptides that Bind PrPc and PrPsc from Scrapie-Infected Hamster Brain Homogenate

One use of the methods described herein is the identification of ligands that preferentially bind to and thereby allow detection and separation of normal versus infectious forms of the prion protein PrPc and PrPsc. Different biochemical properties of PrPc and PrPsc and the binding of antibodies, i.e., 3F4 monoclonal antibody (Signet Laboratories, Inc., Dedham, Mass.) were exploited to find ligands that selectively bind to PrPsc. The monoclonal antibody 3F4 binds to denatured PrPsc and PrPc with considerably higher affinity than non-denatured PrPsc.

A. Peptide Library

Mono- di-, and trimer, peptide libraries were synthesized by Peptides International (Lexington, Ky.) directly on Toyopearl™ 650-M amino resin (Tosoh BioScience, Montgomeryville, Pa.) using standard Fmoc chemistry based on methods described by Buettner et al. 1996. Tetra-, penta- and hexamer peptide libraries included an epsilon amino caproic acid spacer between the amino group and the generation of the library. Peptide densities achieved with the above scheme were typically in the range of 0.1-0.5 mmole/gram dry weight of resin.

B. Protocol for Preparation of Hamster Brain Homogenate (PrP Containing Material)

Ten percent (v/v) homogenates of uninfected and scrapie-infected hamster brains were prepared in phosphate buffered saline, pH 7.4 (PBS) and frozen at −80° C. (courtesy of Dr. Robert Rohwer, VA Medical Center, Baltimore). Prior to use the homogenates were thawed on wet ice and 1.2 ml (uninfected) and 0.5 ml (infected) homogenates were solubilized with 0.5% Sarkosyl with gentle agitation for 30 minutes at room temperature. The samples were centrifuged at 14,000 rpm for five minutes, and the supernatants containing both forms, PrPc (uninfected and infected) and PrPsc (infected only), were collected.

Five milliliters of brain material was prepared by combining 1 ml of normal hamster brain material with 0.33 ml of scrapie-infected brain material and 3.67 ml of Tris-buffered saline (TBS) buffer, pH 7.2, containing 1% casein blocker (Pierce Biotechnology, Inc., Rockford, Ill.) and 1% bovine serum albumin (BSA, Sigma-Aldrich, St. Louis, Mo.). The final ratio of normal to scrapie-infected brain homogenate was three to one, which results in approximately equivalent amounts of PrPc and PrPsc.

C. Protocol for Peptide Library Binding Screening

Five milligrams of dry beads from the peptide library were placed into a Bio-Spin™ disposable chromatography column (Bio-Rad Laboratories, Hercules, Calif.), and were washed with 20 column volumes (CV) of 20% methanol in water to remove possible impurities and organic solvents used in peptide synthesis. The beads were then washed and equilibrated using 20 CV of 1×TBS, pH 7.6 (1×TBS was prepared by 10 fold dilution of 10×TBS (BioSource International, Camarillo, Calif.). The flow was stopped and beads were suspended in 1 ml of fresh 1×TBS and allowed to swell for an additional 15 minutes. TBS was drained out and the column was closed again. To prevent non-specific binding, 1 ml of Blocker™ Casein in TBS (Pierce Biotechnology, Inc., Rockford, Ill.) solution with added 0.5% BSA (Sigma-Aldrich) was applied to the beads. After covering both ends of the column, blocking was performed overnight at 4° C., under gentle agitation. The blocking solution was drained and 1 ml of the hamster brain homogenate prepared above was applied to the resin. The column was tightly closed at both ends and placed in horizontal position and gently agitated at room temperature for one to three hours. The brain homogenate was drained out and beads were washed (gravitationally driven wash) with 10 ml of TBS containing 0.05% Tween 20 (T-TBS), followed by 10 ml of TBS.

D. Protocol for Detection of Bound PrPc

Colorimetric detection of normal PrPc was performed using mouse monoclonal antibody 3F4 (Signet, Dedham, Mass.) diluted 1:8,000 in TBS containing 1% casein. The monoclonal antibody binds native haPrPc, but has little or no affinity for native haPrPsc; however, it does bind denatured haPrPsc and haPrPc. One milliliter of diluted 3F4 antibody was added to previously exposed beads. The column was gently agitated at room temperature for one hour. Antibody containing solution was drained out and beads were washed with 10 ml of TBS and 10 ml of T-TBS. The beads were then incubated in 1 ml of alkaline phosphatase labeled goat anti-mouse secondary antibody (KPL, Gaithersburg, Md.) diluted 1:2,000 in 0.5% casein/0.5% BSA in TBS. Incubation was carried out with gentle agitation for one hour at room temperature. The solution of secondary antibody was drained out and beads were washed with 10 ml of TBS and 10 ml of T-TBS. One milliliter of ImmunoPure Fast Red™ substrate for alkaline phosphatase (Pierce Biotechnology, Rockford, Ill.) was prepared as described by the manufacturer and applied to the beads. Incubation proceeded at room temperature for about 15 minutes, or until beads started turning light pink and a few dark red beads appeared. The substrate solution was drained and the beads washed with 10 ml of TBS. The column was closed at both ends and kept at 4° C. overnight.

E. Protocol for Detection of PrP-Binding Beads Embedded in Agarose

Briefly, the hamster brain homogenate-incubated beads described above were embedded in agarose. First, the base layer of agarose was prepared by covering the surface of a 49 cm² tray (Bio-Rad™ Laboratories, Hercules, Calif.) with 9 ml of 1% agarose (Invitrogen, Carlsbad, Calif.) dissolved in water, which was previously melted and cooled to approximately 40° C. The agarose was allowed to just solidify. Ninety microliters of slurry bead solution at 1.923 mg/ml was added to 800 μl of 0.5% low melting point agarose (SeePlaque GTG Agarose™, FMC BioProducts (now known as Cambrex Bioscience, Inc, Baltimore, Md.) dissolved in water, melted, and cooled to approximately 40° C. The mixture was vortexed briefly and spread over the entire surface of the base layer. A drop of PrP containing material was placed directly into the gel at its the corner and served as a positive control for the next procedures. The gel was allowed to solidify at 4° C. before chemiluminescent detection of PrP-binding beads was undertaken.

F. Protocol for Chemiluminescent Detection of PrP-Binding Beads Embedded in Agarose

After embedding the beads in the gel, a sufficient volume of the chemiluminescent alkaline phosphatase substrate CDP-Star (Tropix Inc. (Applied Biosystems), Bedford, Mass.,) was added to cover the surface of the gel and incubated for five minutes at room temperature as recommended by the manufacturer. The gel was drained of surplus substrate, placed on a clear plastic transparency film, sealed in a plastic bag, and exposed to autoradiography film for 30 minutes. The film (film 1) identified only native PrPc, and beads that bound 3F4 and secondary antibody and subsequently was used to align films obtained after transfer of proteins to a nitrocellulose membrane.

G. Protocol for Protein Transfer from the Embedded Beads to Nitrocellulose Membrane

This methodology elutes proteins from beads and transfers them through capillary action onto nitrocellulose or PVDF membranes. A piece of 3MM filter paper (Schleicher and Schuell, Keene, N H) acts to wick transfer buffer (which can be any buffer that is suited to the particular needs of the experiment) from a tank through the gel in which the beads are immobilized. Accordingly, the 3MM paper wick was pre-wetted with transfer solution and placed on a surface with the ends of the paper immersed in the buffer tank. Six molar (6M) Guanidinium hydrochloride (GuHCl) was used as the transfer solution and was sufficient to dissociate and denature the bound proteins from the beads during the transfer. The gel was placed, bead side up, on the wet 3MM paper, making sure that there were no air bubbles between the paper and the gel. A piece of membrane cut to the size of the gel (ECL-standard nitrocellulose Hybond™ (Amersham Biosciences Corp, Piscataway, N.J.) was wetted in the transfer buffer and placed on top of the gel. A pipette was rolled over the membrane to eliminate air bubbles. Two pieces of pre-wetted 3MM paper were placed on the membrane and rolled with a pipette to remove air bubbles. A stack of dry paper towels or other absorbent paper were placed on top, and weighted with 300 g. Transfer proceeded for 16 hours at room temperature, and resulted in the transfer and immobilization of proteins that were bound to the beads onto the capture membrane.

H. Protocol for ECL (Chemiluminescence) Detection

The membrane onto which the proteins were transferred was placed in a plastic container with 10 ml of 5% (w/v) dried, fat-free bovine milk resuspended in T-TBS (3F4 does not recognize the bovine PrPc present in bovine milk). The membrane was incubated with gentle agitation for 16 hours at 4° C. to prevent non-specific binding of antibodies to the membrane. After blocking, the membrane was incubated with 10 ml of a 1:4,000 fold dilution of primary antibody, 3F4 (Signet), in 5% milk in TBS with gentle agitation for 1.5 hours at room temperature. The primary antibody solution was discarded and the membrane rinsed twice with T-TBS, washed for 15 minutes in T-TBS, then twice for five minutes in fresh T-TBS. All washes were performed with gentle agitation. The membrane was then incubated for 1.5 hours at room temperature with gentle agitation with 10 ml of a 1:10,000 fold dilution of horse radish peroxidase (HRP) labeled goat anti-mouse secondary antibody (KPL) in 5% milk in T-TBS. The secondary antibody solution was discarded and the membranes rinsed and washed as above.

Chemiluminescent detection was accomplished by preparing the HRP chemiluminescent substrate ECL-Plus (Pierce) according to the manufacturer's instructions. Ten milliliters of the mixture was added to each membrane, protein side up. The substrate was gently swirled by hand for one minute and the substrate-saturated membranes removed and placed on 3MM filter paper to drain quickly, then wrapped in a sheet protector (Boise Cascade Office Products, Boise, Ill.). The protein side of the membranes was contacted with autoradiography film for various times and the films developed (film 2).

I. Detection of Trimer-Binders Specific for PrPsc from Scrapie Hamster Brain

The above protocol resulted in production of a gel with a percentage of beads that were stained red, indicating that they bound native PrPc or secondary antibody, a first film with a signal from those beads, and a second film with signals from beads that bound both native PrPc and/or secondary antibody (stained red on the gel) and denatured PrPc and PrPsc, and/or secondary antibody. Upon alignment of the spots on films 1 and 2 with the previously stained beads, four populations of beads were possible: 1) those that bound 3F4 would be stained red and would produce a signal on films 1 and 2; 2) those that bound both PrPc and PrPsc would be stained red and would produce a signal on films 1 and 2; 3) those that bound PrPc alone would be stained red and would produce a signal on films 1 and 2; and 4) those that bound only or preferentially PrPsc would produce a signal on film 2, but would not be stained red, nor would they produce a signal on film 1. This alignment and selection is presented diagrammatically in FIG. 2. The fourth group of beads was selected as PrPsc specific beads. Representative beads from the trimer library that did not produce the signal at the first chemiluminescent detection (film 1, before denaturing step) but produced the signal at the second chemiluminescent detection (film 2, after denaturing step), were sequenced. Several ligand amino acid sequences identified in these and other experiments (including experiments wherein PK was used) are listed in Table 1 below. Several grams of DVR resin were synthesized.

TABLE 1

Peptide that bind PrPc and PrPsc from scrapie-infected
hamster brain homogenate (na indicates 2-naphthyl-
alanine).

| | | Intensity of |
|---|---|---|
| | | Chemiluminescent |
| Sequence | Bead Color | Signal after Denaturing |
| YID (SEQ ID NO:1) | Bright pink | Strong |
| RWD (SEQ ID NO:2) | Bright pink | Strong |
| DVR (SEQ ID NO:3) | White | Strong |
| RES(na)NVA | White | Strong |
| (SEQ ID NO:4) | | |
| ES(na)PRQA | White | Strong |
| (SEQ ID NO:5) | | |
| VARENIA (SEQ ID NO:6) | White | Strong |
| RWEREDA (SEQ ID NO:7) | Pink | Strong |
| EWWETV (SEQ ID NO:8) | White | Medium |
| SVYQLDA (SEQ ID NO:9) | White | Medium |
| (na)HEFYGA | White | Medium |
| (SEQ ID NO:10) | | |
| HE(na)(na)LVA | White | Medium |
| (SEQ ID NO:11) | | |
| SS(na)KKDA | White | Medium |
| (SEQ ID NO:12) | | |
| R(na)DKEAA | White | Medium |
| (SEQ ID NO:13) | | |
| FQGTREA (SEQ ID NO:14) | White | Strong |
| TGTNRYA | White | Strong |
| (SEQ ID NO:15) | | |
| KWATRYA | White | Strong |
| (SEQ ID NO:16) | | |
| NSTKFDA (SEQ ID NO:17) | Pink | Strong |
| EHATYRA | White | Strong |
| (SEQ ID NO:18) | | |

Five milligrams (5 mg) of DVR (SEQ ID NO:3) and Amino 650-M (as a control) were incubated with 1% spCJD brain homogenate, solubilized with 0.1% Sarkosyl, for one hour at room temperature. The presence of PrPc bound to the beads was detected with Fast-Red as previously described. The beads were then immobilized in an agarose gel, detected on the first support, transferred with GuHCl, and detected on the second support. The DVR beads were white under the microscope following on-bead detection and the signal on the first support was weak, indicating little binding of PrPc. The Amino beads were pink and the signal on the first support was strong, indicating that the Amino resin binds PrPc. Following denaturation and transfer, the signal from the DVR (SEQ. ID NO 3) beads on the second support was strong. The signal from the amino beads was also strong, indicating that it binds PrPc, and may also bind PrPsc. These results indicate that DVR (SEQ ID NO:3) preferentially binds PrPsc and confirm that the method can identify ligands that preferentially bind different isoforms of proteins.

### EXAMPLE 2

### Detection of Binders from Trimer-Library Specific for PrPres from Sporadic CJD Brain after Proteinase K Treatment

In this example a trimer library was screened for PrPsc binders from brain homogenate prepared from a patient with human sporadic CJD, and the beads were treated with proteinase K (PK) before the immunodetection of PrPsc-specific binders. The experiment was performed according to the procedures described in the previous example with the following changes: 1) 10 mg of resin per column was incubated with 1 ml of 1.0% brain homogenate diluted into CPD buffer (citrate, phosphate, dextrose (Baxter Healthcare/Fenwal, Deerfield, Ill.) and containing 0.05% Sarkosyl (Sigma-Aldrich) and 0.2 mM of phenylmethanesulfonyl fluoride (PMSF, Sigma-Aldrich); 2) following detection with ImmunoPure Fast Red™ substrate, the beads were incubated with 1 ml of PK (100 μg/ml) at 37° C. for one hour. The result of the PK treatment was the digestion of PrPc before transfer, leaving only PrPres on the beads and the subsequent membrane. This results in film 2 having only the signal generated by 3F4 recognizing PrPres. Alignment of film 2 with the gel containing the beads indicated those beads that are specific for PrPsc. The sequences obtained from this screening were FPK (SEQ ID NO:19), HWK (SEQ ID NO:20), WEE (SEQ ID NO:21), and LLR (SEQ ID NO:22).

Although methods and materials similar or equivalent to those described herein can be used in the practice or testing of the present invention, suitable methods and material are described above. All publications, patent applications, patents and other cited references mentioned herein are incorporated by reference in their entirety. In addition, the materials, methods, and examples are illustrative only and not intended to be limiting.

The foregoing description is provided for describing various embodiments relating to the invention. Various modifications, additions and deletions may be made to these embodiments and/or structures without departing from the scope and spirit of the invention.

1. A method of identifying a ligand having binding specificity a protein isoform in a sample, comprising:
a. contacting one or more ligands with the sample containing at least two protein isoforms under conditions allowing formation of one or more complexes between the one or more ligands and one or more protein isoforms;
b. contacting the one or more complexes with a first detectable marker under conditions allowing generation of a first detectable signal and detecting a presence or absence of the first detectable signal;
c. transferring the one or more protein isoforms from a first support to a second support;
d. contacting the transferred isoforms with a second detectable marker under conditions allowing generation of a second detectable signal and detecting a presence or absence of the second detectable signal;
e. comparing the first signal to the second signal, wherein the presence or absence of the first signal and the presence or absence of the second signal identifies the one or more ligands having binding specificity for the one or more protein isoforms.

2. The method of claim 1, wherein the one or more protein isoform is an isoform of a prion protein.

3. The method of claim 1, wherein the one or more protein isoform is an isoform of a prion protein selected from the group consisting of PrPc, PrPsc, PrPr, and PrPres.

4. The method of claim 1, wherein the one or more protein isoform is a PrPsc isoform of a prion protein.

5. The method of claim 1, wherein the first and the second detectable markers are antibodies.

6. The method of claim 1, wherein the first and the second detectable markers detect different isoforms of a same protein.

7. The method of claim 1, wherein the ligand is immobilized directly on the first support.

8. The method of claim 1, wherein the ligand is immobilized indirectly on the first support.

9. The method of claim 1, wherein the ligand is bound to a solid phase that is immobilized on the first support.

10. The method of claim 1, wherein the one or more ligands are immobilized on the first support prior to contacting the one or more ligands with the sample containing the one or more protein isoforms.

11. The method of claim 1, wherein the one or more ligands are immobilized on the first support after contacting the one or more ligands with the sample containing the one or more protein isoforms and formation of the one or more complexes.

12. The method of claim 1, wherein the one or more protein isoforms are modified to form one or more different protein isoforms after the complex is contacted with the first detectable marker, and the one or more different protein isoforms are contacted with the second detectable marker.

13. The method of claim 12, wherein the one or more protein isoforms are modified by digestion.

14. The method of claim 12, wherein the one or more protein isoforms are modified by denaturation.

15. The method of claim 12, wherein the one or more protein isoforms are modified to form the one or more different protein isoforms before, during or after transfer to the second support.

17. The method of claim 1, wherein the ligand identified as having binding specificity for the one or more protein isoform is a peptide having an amino acid sequence selected from the group consisting of SEQ ID NOS:1-22.

18. The method of claim 1, wherein the ligand identified as having binding specificity for the one or more protein isoforms is a peptide having the amino acid sequence SEQ ID NO:3 or its analogs.

19. The method of claim 1, wherein the ligands comprise a plurality of ligands that form an array on the first support.
